(19) United States
(12) Patent Application Publication
(10) Pub. No.: US 2004/0204645 A1
(43) Pub. Date: Oct. 14, 2004

(54) Scope position and orientation feedback device

(76) Inventors: Vahid Saadat, Saratoga, CA (US); Eugene Chen, Carlsbad, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/411,546

(22) Filed: Apr. 10, 2003

(51) Int. Cl. A61B 5/05; A61B 1/00

(52) U.S. Cl. 600/424; 600/117

(57) ABSTRACT

Methods and devices are disclosed, for mapping the progress of a medical device such as an endoscope within the body of a patient. In one implementation, a flexible display is positioned across the abdomen of the patient. Signals are received by the display from the endoscope, and converted into visual indicium of the location of the endoscope. The display reveals a visible map of the progress of the endoscope, in real time, which may be in 1:1 size correspondence with the path of the endoscope.

### BACKGROUND OF THE INVENTION

1. Field of the Invention

The present invention relates to the field of endoscopy, and more particularly, to the sensing and displaying of the position and orientation of an endoscope within a patient.

2. Description of the Related Art

During endoscopic procedures, such as colonoscopy, for example, it can be very difficult for a technician to ascertain the position and orientation of a scope within a patient. This is due, in large part, to the highly flexible nature of the scope being inserted and pushed into a patient's colon, which has unpredictable fixation points to the viscera of the abdomen and is easily distensible. Moreover, the scope is highly flexible and many types of scopes are also free to bend as they encounter the colon wall. This problem is exacerbated when using an endoscope having a steerable tip. For example, once the steerable tip is deflected to enter a new area of the intestinal tract, the principal direction of force urging the endoscope to advance is no longer in the direction of the steerable tip. Instead, the force is directed along the axis of the endoscope and causes yielding or displacement of the colon wall.

Alternatively and many times in addition, the endoscope is typically free to bend and flex in response to external forces applied resulting from encounters with the colon wall. For example, especially in minimally supported parts of the anatomy, such as the gastrointestinal tract (“GI tract”), it is common for a highly flexible scope to form an alpha loop as it is advanced. That is, as a proximal end of the scope, which usually comprises a handle, is used to push the flexible distal portion of the scope into a patient, the distal end will form a loop rather than progress further through the GI tract.

Consequently, while a technician can easily verify the length of the inserted portion of the scope, a technician is generally unable to determine the precise location, path, or orientation of the scope.

To this end, a variety of imaging techniques have been used to visualize the location of a scope within a patient. For example, x-ray technology has been widely used to display a scope within a patient. However, this type of visualization technique is expensive in terms of equipment and time. Furthermore, a patient, technician, and other hospital personnel may be exposed to sustained periods of harmful x-radiation.

Alternatively, fluoroscopy procedures have been commonly used to visualize the position and curvature of the scope. However, several drawback, including expensive and bulky equipment, scarcity of equipment and trained technicians, and most importantly, x-radiation exposure, prevent this from being a simple and efficient solution.

Another approach employs the use of magnetic fields. One such approach establishes a low frequency magnetic field around the patient and a miniature sensor embedded within the scope is tracked sequentially and the data is then used to display a representation of the scope tube configuration on a remote monitor. However, it has been found that distortions, some of which may be caused by the scope materials, prevent this from being a very accurate representation. Moreover, the cumbersome equipment required to establish the low level magnetic field is placed over, and within close proximity to, the patient and can interfere with the endoscopic procedure. Additionally, non-metallic operating tables, instruments, and other required devices are necessary so as to avoid causing distortions within the magnetic field that can be misinterpreted by the display thus resulting in an inaccurate representation of the scope orientation.

Yet another approach is to sense the curvature of the scope electrically. This approach utilizes a plurality of strain gages placed along the scope which alter their electrical resistance in response to the strain each sensor experiences as the scope is manipulated. Typically, one or more Wheatstone bridges are used to detect the signal resulting from the change in resistance of the strain gage as the scope deflects. However, these type of systems can be sensitive to climatic changes, such as temperature and moisture. Moreover, they can be difficult to manufacture and require complicated equipment to receive and translate their signals.

In view of the above, an important need remains in the art for a scope position and orientation sensor and display that can quickly, efficiently, and accurately indicate the spatial orientation of a scope positioned within a patient.

### SUMMARY OF THE INVENTION

There is provided in accordance with one aspect of the present invention, a method of tracking the spatial orientation of a device within the body of a patient. The method comprises the steps of introducing a device into the body, the device carrying at least one signal source thereon. A display is provided, having a first side for facing the patient and a second side for displaying indicium of the location of the signal source. The first side is positioned such that it faces the patient, and an indicium of the location of the signal source is displayed on the second side.

The introducing step may comprise introducing an endoscope into the body. The positioning step may comprise positioning the first side in contact with the patient. The positioning step may additionally comprise forming the first side into a nonplanar configuration to conform to the surface of the patient. The positioning step may additionally comprise positioning the first side such that it faces the patient's abdomen.

The displaying step may comprise displaying a line which approximates the shape of the path of the device within the patient. The displaying step may alternatively comprise displaying at least a first point and a second point which coincide with the path of the device within the patient. The size of the displayed indicium and the size of the path of the device within the body may be in approximately a 1:1 relationship.

There is provided in accordance with another aspect of the present invention, a spacial orientation sensor for sensing the location within the body of at least one signal source. The spatial orientation sensor comprises a flexible pad having a first side for contacting the patient and a second side for displaying indicium of the location of the signal source. The indicium may comprise light emitting diodes. The indicium may display the actual X and Y axis location of the signal source, and may be vertically displaced from the signal source in the Z direction.

The spatial orientation sensor may further comprise a plurality of receivers for sensing the location of the signal source. The sensor may additionally comprise a control unit for analyzing the signals sensed by the receivers, determining the location of the signal source, and activating displaying indicium corresponding to the location of the signal source. The control unit may include a central processing unit for analyzing the signals.

In one implementation, the flexible pad has an area of at least about 1 square foot. In general, the flexible pad has an area within the range of from about 1 square foot to about 9 square feet. The flexible pad may have a thickness of no more than about 2 inches.

The receivers may be capable of detecting ultrasound. Alternatively, the receivers are capable of detecting a radiofrequency signal. Alternatively, the receivers are capable of detecting a magnetic field. Alternatively, the receivers are capable of detecting infrared light. Alternatively, the receivers are capable of detecting ultraviolet light. The detectors may be capable of receiving more than one of the foregoing signals. In one implementation, the flexible pad comprises a radiofrequency identification tag transponder.

In accordance with further aspect of the present invention, there is provided an endoscope tracking system. The tracking system comprises an array of sensors carried by a support. A display is also carried by the support. An endoscope, carrying a plurality of signal sources thereon, is provided for use in conjunction with the support.

The array of sensors is configured to detect signals emitted from the plurality of signal sources. The signals may be selected from the group consisting of radiofrequency, ultrasound, magnetic, light or radioactivity, or combinations thereof. The signal sources may be removably carried by the endoscope. Alternatively, the signal sources are attached to the endoscope. The signal sources may be spaced axially apart along a tracking zone of the endoscope. The tracking zone may have an axial length of at least about 50 cm, preferably at least about 20 cm, and generally within the range of from about 10 cm to about 100 cm for lower GI applications.

The signal sources may generate a signal in response to an input signal carried through the endoscope. Alternatively, the signal sources may originate a signal, without the need for an input signal. The signal sources may alternatively generate a signal in response to receipt of an interrogator signal. An interrogator signal source may be carried by the support. Alternatively, an interrogator signal source may be carried remotely from the support.

The signal may be constant throughout the medical procedure. Alternatively, the signal may be pulsatile during the medical procedure.

In accordance with a further aspect of the present invention, there is provided a method of displaying the location of a medical device within a body. The method comprises the steps of introducing a medical device into the body, and propagating at least a first signal from the medical device. The signal is received by a receiver outside of the body, and an indicium of the signal is displayed. The displaying step comprises displaying an indicium which corresponds with the actual location of the origin of the propagated signal, in approximately a 1:1 size relationship, at least in the X and Y axes. In one implementation of the invention, the display does not display Z axis location information. The displaying step may comprise displaying the indicium on a flexible pad. The flexible pad may be in contact with the body. Alternatively, the displaying step may comprise displaying the indicium on a display that is positioned remotely from the body.

In accordance with another aspect of the present invention, there is provided a detection device for detecting the location of a signal source within the body. The detection device comprises a support having a plurality of detectors thereon. A first plurality of detectors generate a first signal indicative of the proximity of the signal source, which is distinguishable from any signal generated from a second plurality of detectors which are more remote from the source.

In one implementation, the first signal is a visible signal. The device may additionally comprise a display, carried by the support, for displaying the first signal.

Further features and advantages of the present invention will become apparent to those of ordinary skill in the art in view of the detailed description of preferred embodiments which follows, when considered together with the attached drawings and claims.

### BRIEF DESCRIPTION OF THE DRAWINGS

FIG. 1 is an isometric view showing one embodiment of a scope having a plurality of built-in signal generators.

FIG. 1A is a cross-sectional view of the scope taken along the line 1A-1A in FIG. 1.

FIG. 2 is an isometric view showing another embodiment of a signal generator used in conjunction with a typical endoscope.

FIG. 3 is an isometric view illustrating yet another embodiment of a signal generator attached to a scope sheath.

FIG. 4 is an isometric view showing a plurality of signal generators placed within an external channel of a scope.

FIG. 5 is an isometric view illustrating one embodiment of a of plurality signal generators attached to a clip configured for attachment to a scope.

FIG. 6 is an isometric view of an alternative embodiment of a signal generating scope clip.

FIG. 7 is a schematic illustration showing a plurality of jointly connected signal generators for simultaneous activation.

FIG. 8 is a schematic illustration showing a plurality of jointly connected signal generators for controlled activation.

FIG. 9 is an isometric view showing one embodiment of a flexible support having a plurality of sensors and a plurality of displays.

FIG. 10 is a cross-sectional view taken along line A-A of FIG. 9 showing one embodiment of the plurality of sensors and displays carried by the flexible support of FIG. 9.

FIG. 11 is a cross-sectional view taken along line A-A of FIG. 9 support another embodiment of the plurality of sensors and displays carried by the flexible support of FIG. 9.

FIG. 12 is an isometric view of another embodiment of a flexible support having three sensors and a plurality of displays.

FIG. 13 is an isometric view showing an example of a plurality of activated displays carried by a flexible support.

### DETAILED DESCRIPTION OF THE PREFERRED EMBODIMENTS

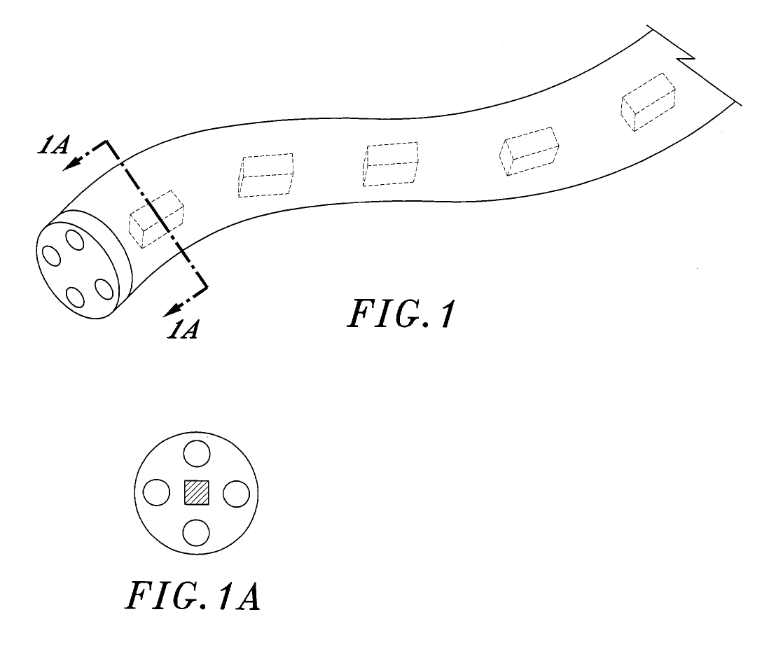

With reference to FIG. 1, an endoscope 10 is an elongate flexible member having a diameter of about 1 cm and a length of between about 50 cm and 200 cm. The scope may be made of any suitable biocompatible material, such as, for example, polyvinylchloride, polyurethane, or polyethylene. The endoscope includes a plurality of conduits or lumen 12 formed longitudinally therethrough for carrying illumination devices, visualization devices such as cameras, and biopsy or other diagnostic or therapeutic surgical tools. Many of these devices are permanently carried within their corresponding conduits; however, a working channel 18 is normally provided for the selective insertion and removal of specialized surgical or other tools as desired.

The present invention will be primarily described herein in the context of an endoscope for lower gastrointestinal applications. However, it should be apparent to those of skill in the art that the present invention has much greater applicability, and may be readily adapted for use in tracking any of a wide variety of medical devices throughout the body. For example, medical devices incorporating the tracking features of the present invention may be utilized in any of a wide variety of applications involving propagation of a device through a naturally occurring body lumen or hollow organ, or through a surgically created incision or tissue pathway. In the context of endoscopic applications, the present invention may be incorporated into a wide variety of diagnostic and/or therapeutic endoscopes such as endoscopes for the upper esophagus, stomach, duodenum, angioscopes for blood vessels, bronchoscopes for the bronchi, arthroscopes for joint spaces, colonscopes for the colon, and laparoscopes for any of a wide variety of lapascopic procedures, primarily within the peritoneal cavity. The tracking features of the present invention may alternatively be incorporated into a wide variety of tools for minimally invasive surgical procedures, as will be apparent to those of skill in the art in view of the disclosure herein.

The illustrated embodiment further comprises one or more signal sources, or transducers 14, positioned within the scope at discrete positions along its length. As used herein, the terms “signal source” and “transducer” are used interchangeably to refer to any device that emits a detectable signal. Examples include, but are not limited to, magnetic, radio frequency (RF), ultra sound (UTZ), and other suitable signal source generators.

Certain signal sources such as permanent magnets may generate a continuous signal independent of any control signal. Magnetic fields may also be generated by electromagnets, which generate a magnetic signal in response to the introduction of an electrical current, which may be carried from a power source at the proximal end of the endoscope through conductive elements extending through the endoscope. Other sources such as radio frequency and ultrasound sources may generate a signal in response to receipt of an electrical impulse carried by way of electrical conductors extending from a proximal control through the endoscope. A further class of signal sources include sources which generate a signal in response to receipt of an interrogation signal from an interrogator signal source. Signal sources of this type are well understood in the radio frequency identification tag arts, and need not be disclosed in detail herein. In general, an interrogator signal may be propagated from the patient interface or pad, discussed below. Alternatively, the interrogator signal may be propagated from a device which is remote from the display pad, as will be understood by those of skill in the art in view of the disclosure herein.

In the illustrated embodiment, the transducers 14 are integrally formed within the scope 10 and may be spaced at any suitable interval, such as, for example, at least about 0.1 cm, or at least about 1 cm, or at least about 2 cm, or at least about 5 cm, or at least about 10 cm apart. In one embodiment, the transducers 14 are spaced approximately 0.5 cm apart, throughout a tracking zone on the medical device.

Depending upon the nature of the medical device to be tracked, a larger or smaller portion of the axial length of the device may desirably be visualized using the scope position and orientation feedback device of the present invention. For example, an endoscope having an axial length of approximately 200 cm for use in the colon, may have a tracking zone within the range of from about 50 cm to about 150 cm in length. A proximal transducer 14 is provided at a proximal end of the tracking zone and a distal transducer 14 is provided at a distal end of the tracking zone. A plurality of transducers 14 may be spaced apart throughout the length of the tracking zone.

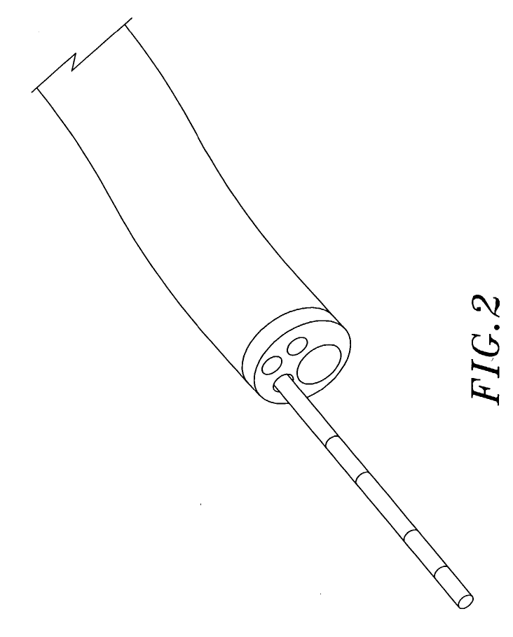

The transducers 14 can be driven electrically, such as by wires extending through the scope and connected to a suitable source of power. Such a transducer 14 is configured to produce a sufficiently powered signal to transmit through surrounding tissue and adjacent device materials. Alternatively, the transducers 14 may inherently emit a signal source, such as a magnetic field, or a particle emission such as during decay. In any case, the transducers 14 function as a signal source that emit a detectable signal. The following description further describing FIGS. 2-6 discuss alternative embodiments of transducer 14 configuration and mounting locations.

In one embodiment, UTZ transducers emit ultrasound at a specific frequency that falls within a specified bandwidth, such as about 200 kHz to about 15 MHz. Alternative embodiments utilize UTZ transducers that can be differentiated by unique signatures. For example, a plurality of transducers 14 can be configured to each emit a signal at a unique frequency. Alternatively, each transducer can emit a common frequency but vary the pulse length or pulse burst sequences to allow individual transducer 14 identification. Such a transducer 14 can thus provide a unique signal that allows it to be individually identified. In many embodiments, there is preferably a control unit configured to control the transmitting strategy of the various transducers 14, and will be discussed later in detail.

The control unit may include a central processing unit for interpreting the data received by the transducer 14, and driving the display. The central processing unit may be programmed to make assumptions which allow it to fill in space between adjacent transducers, such as to provide a continuous visual illustration of the progress of the endoscope. The central processing unit may additionally be programmed to differentiate signals of different strength, and signals from noise, in order to interpret data from a plurality of transducers and produce a final illustration of the location of the medical device. Programming and implementation details of this nature are within the exercise of routine skill in the art, given the desired clinical performance of the device, as will be apparent from the disclosure herein.

FIG. 1A illustrates a cross-sectional view of the endoscope of FIG. 1 showing the plurality of conduits 12 and one possible position of a transducer 14 permanently installed during endoscope manufacture. Of course, the transducers 14 could be placed at any location within or along the endoscope 30 so long as they don't interfere with the operation of the longitudinal working channels 12.

FIG. 2 illustrates an embodiment of an endoscope 10 in which one of the longitudinal channels 12 receives an elongate flexible rod 16 carrying one or more transducers 14. In the illustrated embodiment, the elongate rod 16 may be a wire, a rod, a catheter, or any other suitable elongate member for slideable insertion into an endoscope working channel 18. The elongate rod is preferably longer than the endoscope 10 such that the rod may be inserted from a proximal end of the endoscope and advanced to about the distal end of the scope 10. In one embodiment the elongate rod 16 may be securely fixed relative to the endoscope 10 such that, once properly inserted into the working channel 18, undesired longitudinal movement is inhibited. Additionally, the insertable elongate rod 16 may be selectively removed once the endoscope 10 is appropriately positioned and other tools may be subsequently advanced through the working channel 18.

Accordingly, the plurality of transducers 14 may be carried by a flexible support which is integrally formed with the endoscope 10 or other medical device, or removably coupled to the medical device. This allows a removable support such as flexible rod 16 to be removed and sterilized for reuse. Alternatively, the flexible support carrying transducers 14 may comprise a one-time use disposable component. In an embodiment such as that illustrated in FIG. 2, with a flexible rod 16 adapted to be positioned within a working channel 18, the proximal end of the rod 16 may be provided with any of a variety of luer connectors or other removable connectors, for locking the rod 16 within the endoscope during the medical procedure. In this manner, the working zone on the rod 16 can be axially indexed with the appropriate corresponding structure on the endoscope 10, to allow proper visualization of the progress of the endoscope 10.

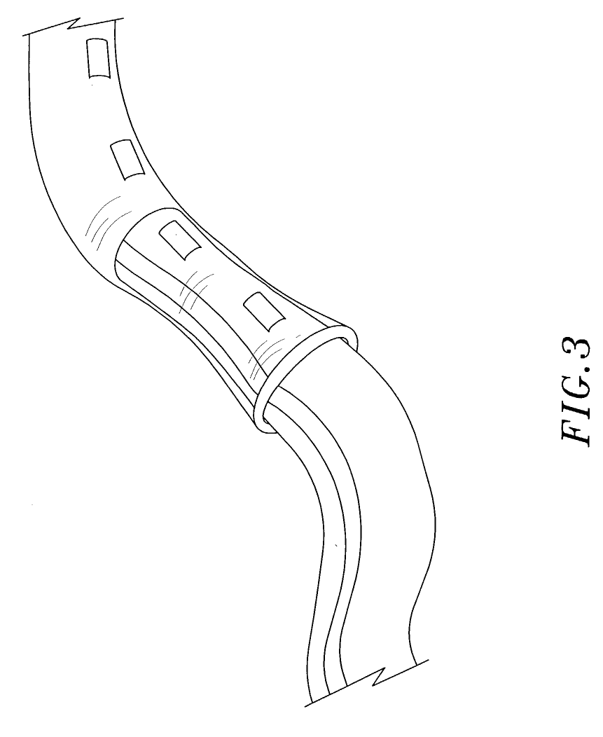

FIG. 3 illustrates another embodiment of the present invention in which a plurality of transducers 14 are mounted within or to an external sheath 20 which may be slideably disposed over the endoscope 10. The sheath 20 may be any suitable sheath for covering an endoscope. As discussed above, the transducers 14 may be any suitable transducers capable of emitting a detectable signal and may either be powered via electrical wires, or may inherently emit a signal.

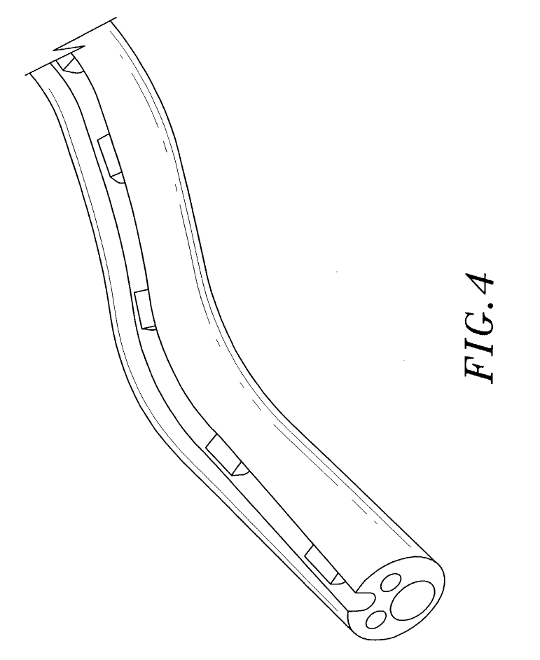

FIG. 4 illustrates another embodiment of an endoscope 10 that has an external channel 22. With this type of scope, a plurality of transducers 14 can be mounted within the external channel 22 of the scope. It is preferable, however, that the transducers 14 are fully contained within the external channel 22 such that the transducers 14 do not extend beyond the outer circumference of the endoscope 10 and interfere with the insertion, manipulation, or operation of the scope.

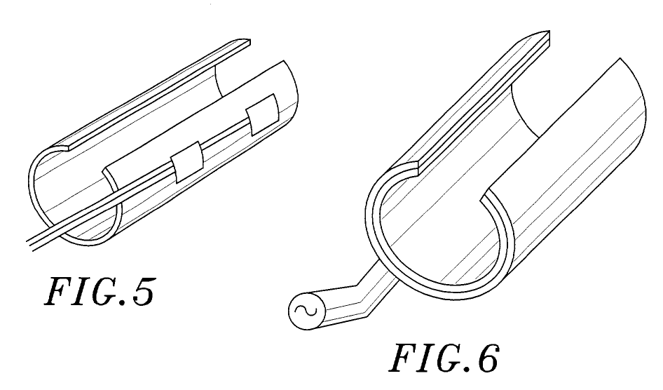

FIGS. 5 and 6 illustrate embodiments of a clip 24 designed to be attached to the outer periphery of an endoscope somewhere along its length. The clip may be formed of any suitable resilient material but is preferably formed of biocompatible materials such as polyvinylidene fluoride (PVDF). The clips 24 are substantially C-shaped in cross section, and can be elastically deformed to fit around the circumference of a scope 10. Preferably, the clips 24 are biased such that the clip 24 will return substantially to its C-shaped cross section once it has been attached to the scope 10. The clip 24 may be maintained upon the scope 10 by friction between the scope 10 and the clip 24. Additional friction enhancing features may be added, such as surface features of the clip 24 and/or scope 10 such as ridges, surface roughness, appropriate glutinous substances, or any other suitable method for increasing the friction between the clip 24 and the scope 10. Alternatively, the clip 24 and scope 10 may be configured with cooperating structure for maintaining the relative position and/or to effectuate mounting of the clip 24 to the scope 10.

In the illustrated embodiment of FIG. 5, one or more transducers 14 are attached to the clip 24. The transducers 14 may be attached by any suitable method such as adhesives, welding or other mechanical or chemical bonds. Moreover, the transducers 14 may be permanently or temporarily attached to the clip 24. The transducers 14 are connected together and to a control unit (not shown), by wires.

With specific reference to FIG. 6, a scope clip 24 can be formed of suitable materials such that the clip 24 itself emits a signal suitable for detection by a receiver as discussed hereinafter. One example of such a clip 24 may be formed of piezocomposite materials, which in one embodiment comprises a combination of PVDF and ceramic piezo crystals, formed into a clip 24 for attachment to the outer periphery of the scope. The materials can be layered as illustrated, with the PVDF layer 26 serving as a substrate for the ceramic piezo crystal layer 28. A driver 32 is connected to the clip 24, such as by wires 34, for providing the necessary power requirements and controlling the desired signal output. In this type of embodiment, the clip 24 emits an appropriate ultrasonic signal which can then be detected as described hereinafter.

A plurality of clips 24 may be spaced along the scope 10, and each clip 24 may exhibit unique signal characteristics to allow one clip 24 be distinguished from other clips 24. Alternatively, each transducer 14 on each clip 24 may exhibit unique signal characteristics such that each transducer 14 can be individually identified.

With reference to FIG. 7, a plurality of transducers 14 can be commonly connected such that activation of a single transducer 14 results in the activation of the plurality of transducers 14. The illustrated embodiment depicts a plurality of transducers 14 connected in parallel. An appropriate driver 32 provides the necessary power and can additionally provide a control strategy for activation of the transducers 14. The transducers 14 may be tuned to emit a common frequency, or can be configured to transmit unique signals that can serve to individually identify each transducer 14.

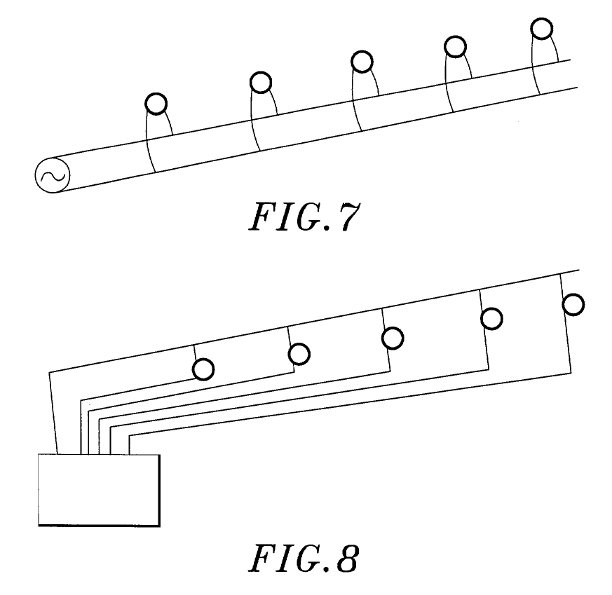

With reference to FIG. 8, a control unit 38 is provided to drive the transducers 14 according to a desire control strategy. In this embodiment, each transducer 14 is individually wired to the control unit 38 and operates according to any of a number of selected modalities. For example, the control unit 38 may be programmed to activate each transducer 14 sequentially, simultaneously, or in any other desired modality. Furthermore, the control unit 38 can vary characteristics of the signal source such as frequency, amplitude, pulse length, or pulse burst sequence to provide a unique signal for each transducer 14.

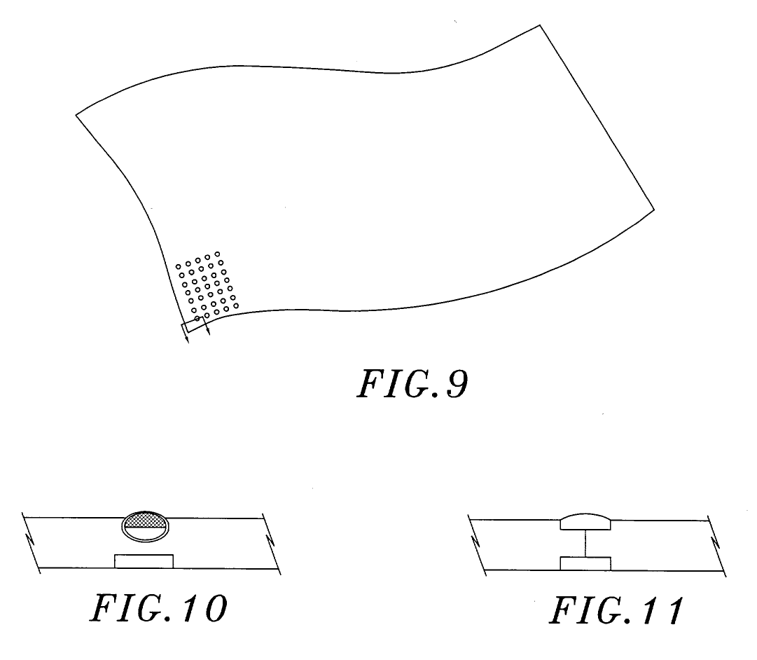

With reference to FIG. 9, a plurality of display devices 40 are carried by a flexible support member 42, or display. The illustrated embodiment shows the flexible support member 40 as a relatively thin elastomeric sheet. The support member 40 may be of any desired size, but in one embodiment, is about 91 cm (36 in.) by 61 cm (24 in.). It may be of any desired thickness, but in one embodiment, is about 1 cm (0.4 in.) thick. In one embodiment, the support member is formed of any suitable flexible material, such as, for example, any of a number of polyethylenes, urethanes, neoprenes, silicones, and the like. Other suitable textile or composite materials may equally be suitable as a flexible support 42 and are contemplated as within the scope of the present invention.

The display devices 40 are carried by the flexible support member 40 in any suitable manner and function to indicate the proximity of a transducer 14 attached to a scope 10. With additional reference to FIGS. 10 and 11, one side of the flexible support 42 carries a plurality of display devices 40, while the opposing side of the flexible support 42 carries a plurality of receivers 44.

With specific reference to FIG. 10, a display device 40 may be color-coded such that one side is distinguishable from the other side. The illustrated embodiment shows a sphere in which each hemisphere is color-coded to contrast with one another. In this particular embodiment, the flexible support is approximately one centimeter thick and each of the display devices are held by an appropriately configured holder 46 within the display device 42. Each spherical display device 40 is held within a concave depression 46 that is configured to securely hold each spherical display device 40 within the flexible support 42 while allowing rotational movement of the spherical display device 40 within the holder 46.

Such a spherical display device 40 may be formed of any suitable material, such as plastic, ceramic, or metal. Each spherical display device 40 is configured to respond to a signal emanating from one or more transducers 14 carried by the scope 10. For example, each spherical display device 40 could be magnetized with a magnetic pole associated with each colored hemisphere. A transducer 14 can establish a magnetic field to which the spherical display device 40 could respond by orienting the appropriate pole toward the magnetic field source. By incorporating a plurality of spherical display devices 40 at appropriate intervals along and across the display 42, an approximate representation of the position of the transducers 14 carried by the scope 10 can be generated.

Alternatively, a receiver 44 can be associated with each spherical display device 40 and can generate a localized magnetic field in response to signals received from proximate transducers 14. For example, a plurality of transducers 14 each emit a signal that is received by a plurality of receivers 44 mounted on the flexible support 42. A control unit (not shown) can interpret the signals received by the receivers 44 and compare the relative signal strengths of each receiver 44 to determine which receivers 44 are in closest proximity to the active transducer 14. The control unit can then instruct the appropriate receivers 44 to either signal or control the display device 40 to indicate the presence of the transducer 14.

In the embodiment of FIG. 10, the receiver 44 could establish a local magnetic field that would cause the spherical display device 40 to orient itself in a way that signals to a technician the approximate position of a transducer 14 within a patient. A plurality of spherical display devices 40 would thus all signal to a technician the approximate position of each transducer 14, and would thus display the approximate spatial orientation and position of the scope 10 within a patient's body.

With particular reference to FIG. 11, the display device 40 may be in the form of a light-emitting diode (LED), fiber optic cables, or other light emitting device 48 that may be activated by a corresponding receiver 44 in response to an appropriate signal. As described above, each transducer 14 emits a signal that is received by one or more receivers 44. A control unit can then compare the received signals and determine which receiver 44 is closest in proximity to each transducer 14. Finally, the receiver 44 can activate the display device 40 to emit light thereby indicating the approximate position of each transducer 14. The intensity of the display device 40 can be proportional to the intensity of the signal received by the corresponding receiver 44. As such, the display devices 40 closer to the signal source will have a higher intensity light.

The nature of the display can be varied considerably, depending upon the desired clinical experience. In one implementation of the invention, the display is carried directly by the flexible support which conforms to the surface of the patient. This puts certain design constraints on the display, which may desirably be a plurality of LED or other discrete light sources, or other flexible display medium. If the display is allowed to be spaced apart from the patient, it may be a rigid structure, and take the form of a more conventional cathod ray tube, LCD display or the like. Flexible display media may also include any of a variety of known chemical systems which exhibit a color change in response to a change in temperature, which may be initiated by heating elements activated in response to interpretation in the CPU of the information received from the transducers 14.

The resolution of the display 42 may be determined by the spacing of the display devices 40, which in some embodiments are spaced at approximately ½ cm, 1 cm, 2 cm, 4 cm, or 5 cm intervals. Accordingly, by having a plurality of active transducers 14 positioned along the length of a scope 10, a plurality of display devices 40 will activate thus indicating an X-Y coordinate path that correspond with the actual path of the scope 10 within the patient.

Additionally, a single receiver 44 may be responsible for activating one or more display devices 40. FIGS. 10 and 11 illustrate a one to one correlation between the number of receivers 44 and the number of display devices 40 with each receiver 44 controlling a single display device 40. However, this is not a requirement of the present invention. By providing at least 2 receivers 44 and 2 transducers 14, at least a linear direction of the scope 10 can be established.

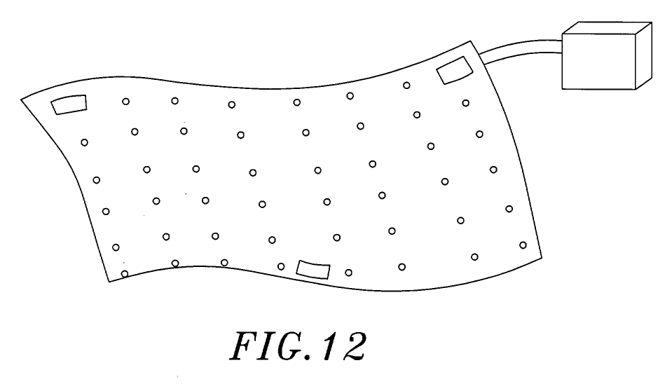

With reference to FIG. 12, a flexible support 42 can comprise three receivers 44 coupled to a control unit 38. The control unit 38 is further coupled to a plurality of display devices 40, which may be of any suitable type. It is preferable that the three receivers 44 are spaced a distance from one another about the flexible support 42.

In the illustrated embodiment, a plurality of transducers 14 on the scope each emit a unique signal that can be detected by each receiver 44. The receivers are under the control of the control unit 38 which analyzes the received signals and determines the signal source direction. By implementing three receivers 44, the control unit 38 can determine the precise direction and/or distance to each transducer 14 by triangulation. Triangulation is the process of determining the location of a single signal source by determining a direction vector from three spaced apart locations to the signal source and then calculating the intersection of those vectors. The vector intersection indicates the location of the signal source.

In the illustrated embodiment, each transducer 14 emits a unique signal identifier, which is received by each of the receivers 44. The control unit 38 determines the respective direction vectors from each receiver 44 to each transducer 14 and then compares the direction vectors received by each of the receivers 44 for each of the signals received from the transducers 14 and determines the location of each transducer 14. Finally, the control unit 38 can activate the display device 40 that closely corresponds with the location of each transducer 44.

Additionally, the control unit 38 can activate additional display devices 40 to result in a higher resolution image. For example, the transducers 14 may be spaced at 5 cm intervals along the scope 10. A simple one-to-one display of the transducer 14 locations would result in display devices 40 being activated at corresponding 5 cm intervals. Under direction of the control unit 38, additional display devices 40 may be activated to result in a more continuous approximation of the scope's spatial orientation.

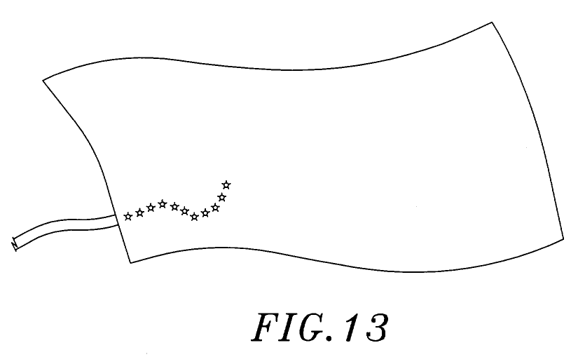

FIG. 13 illustrates one embodiment of the flexible support showing the activated display devices 40. As shown in FIG. 13, the relative position of the scope 10 is approximated by activating the display devices 40 to correspond with the path and orientation of the transducers 14 on the scope. As described above, one embodiment utilizes LEDs. Of course, as described above, other types of display devices 40 may be used to display the approximate orientation and the position of the scope 10.

In use, a technician places the flexible support 42 over a patient. The flexible support may directly contact the patient, or may be suspended above the patient. In either case, the flexible support 42 is generally horizontal and defines an XY plane. A scope 10 configured for use with the flexible support 42 is then inserted into a patient. One or more transducers 14 carried by the scope emit a detectable signal, which signal is detected by receivers 44 carried by the flexible support 42. Preferably, a control unit 38 analyzes the signals received by the receivers 44, and activates one or more display devices 40 that correspond with the approximate position of each transducer 14, thus resulting in a true scale display of the approximate spatial orientation of the scope 10. While the flexible display 42 is typically positioned on, or above, a patient and thus may not provide accurate information in the Z direction, the displayed information represents the actual positions of the transducers 14 in the X and Y directions.

According to another feature, a three-dimensional array of display devices 40 can be positioned above a patient. Based upon signals from a control unit 38, the display devices 40 can produce a three-dimensional representation of the spatial orientation of the scope 10.

The X and Y position can be measured as described above, while the Z position, or depth dimension, can be calculated based upon the signal characteristics, the time measured between signal generation and reception, and the characteristics of intervening tissue and materials. Ultrasound coupling gel may optionally be applied to the patient to provide a more repeatable reception of the signals generated by the transducers 14.

In this embodiment, the support member 42 may be in the form of an inflatable, transparent support blanket having one or more air pockets. The support member 42 may be inflated to a depth of approximately 15 cm (6 in.) to 25 cm (10 in.) or may be up to about 60 cm (24 in.). The display devices 40 are preferably dispersed substantially uniformly throughout the support member 42 in the X, Y, and Z directions. The display devices 40 may be uniformly spaced at any desired resolution, such as, for example, ½ cm, 1 cm, 2 cm, or 5 cm. Of course, other resolutions besides the examples given are possible without departing from the scope hereof.

Alternatively, Z axis information may be at least qualitatively displayed on the LED, LCD or other display. This may be accomplished by a change in the signal intensity to reflect that the medical device is traveling away from or towards the sensor pad. Alternatively, color changes may be utilized on the display to indicate travel of the medical device towards or away from the sensor pad. The desirability of precise, qualitative, or no X axis travel information will be determined in view of the particular clinical application of the invention. If true three dimensional tracking is desirable, and the tracking information is displayed on a LCD or CRT display, the central processing unit may be programmed to allow switching between a display of the top plan view of patient, and a side view of the patient, or to allow split screen simultaneous viewing of a top view and a side view of the progress of the instrument. In this embodiment, the signal will generally be propagated from the receiver pad to the central processing unit and back to the display, so that the central processing unit can interpret the data and display the desired viewing axes.

For example, three or more receivers 44 may be spaced apart and can be attached to the support member 42, or may be external to thereto. The receivers 44 are configured to sense the signals generated by the transducers 14. A control unit 38 can then analyze the signals and determine the location of each transducer 14 in the X, Y, and Z directions. The control unit 38 can then activate the appropriate display devices 40 to generally display a three-dimensional representation of the spatial orientation of the scope 10. Unlike the other embodiments utilizing a two-dimensional display, the three-dimensional display gives an accurate representation of the actual X and Y coordinates, and is spaced above each transducer 14 by a fixed Z coordinate dimension.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or subcombinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the claims that follow.

What is claimed is:

1. A method of tracking the spatial orientation of a device within the body of a patient, comprising the steps of:
introducing a device into the body, the device carrying at least one signal source thereon;
providing a display, having a first side for facing the patient and a second side for displaying indicium of the location of the signal source;
positioning the first side such that it faces the patient; and
displaying an indicium of the location of the signal source on the second side.

2. A method of tracking the spatial orientation of a device as in claim 1, wherein the introducing step comprises introducing an endoscope into the body.

3. A method of tracking the spatial orientation of a device as in claim 1, wherein the positioning step comprises positioning the first side in contact with the patient.

4. A method of tracking the spatial orientation of a device as in claim 3, wherein the positioning step comprises forming the first side into a nonplanar configuration to conform to the surface of the patient.

5. A method of tracking the spatial orientation of a device as in claim 1, wherein the positioning step comprises positioning the first side such that it faces the patient's abdomen.

6. A method of tracking the spatial orientation of a device as in claim 1, wherein the displaying step comprises displaying a line which approximates the shape of the path of the device within the patient.

7. A method of tracking the spatial orientation of a device as in claim 1, wherein the displaying step comprises displaying at least a first point and a second point which coincide with the path of the device within the patient.

8. A method of tracking the spatial orientation of a device as in claim 1, wherein the size of the displayed indicium and the size of the path of the device within the body are in approximately a 1:1 relationship.

9. A spatial orientation sensor, for sensing the location within the body of at least one signal source, comprising a flexible pad having a first side for contacting the patient, and a second side for displaying indicium of the location of the signal source.

10. The spatial orientation sensor of claim 9, wherein the indicium comprise light emitting diodes.

11. The spatial orientation sensor of claim 9, wherein the indicium display the actual x and y location of the signal source and are vertically displaced from the signal source in the z direction.

12. The spatial orientation sensor of claim 9, further comprising a plurality of receivers for sensing the location of the signal source.

13. The spatial orientation sensor of claim 9, further comprising a control unit for analyzing the signals sensed by the receivers, determining the location of the signal source, and activating display indicium corresponding to the location of the signal source.

14. The spatial orientation sensor of claim 13, further comprising a central processing unit for analyzing the signals.

15. The spatial orientation sensor of claim 9, wherein the flexible pad has an area of at least about one square foot.

16. The spatial orientation sensor of claim 9, wherein the flexible pad has an area within the range of from about one square foot to about nine square feet.

17. The spatial orientation sensor of claim 9, wherein the flexible pad has a thickness of no more than about two inches.

18. The spatial orientation sensor of claim 12, wherein the receivers are capable of detecting ultrasound.

19. The spatial orientation sensor of claim 12, wherein the receivers are capable of detecting a radio frequency signal.

20. The spatial orientation sensor of claim 12, wherein the receivers are capable of detecting a magnetic field.

21. The spatial orientation sensor of claim 12, wherein the receivers are capable of detecting infrared light.

22. The spatial orientation sensor of claim 12, wherein the receivers are capable of detecting ultraviolet light.

23. The spatial orientation sensor of claim 12, wherein the flexible pad comprises a radio frequency identification tag transponder.

24. An endoscope tracking system, comprising:
an array of sensors carried by a support;
a display, carried by the support; and
an endoscope, carrying a plurality of signal sources thereon.

25. The endoscope tracking system of claim 24, wherein the array of sensors is configured to sense signals emitted from the plurality of signal sources.

26. The endoscope tracking system of claim 24, wherein the signals are selected from the group consisting of radio frequency, ultrasound, magnetic, light or radioactivity.

27. The endoscope tracking system of claim 24, wherein the signal sources are removably carried by the endoscope.

28. The endoscope tracking system of claim 24, wherein the signal sources are attached to the endoscope.

29. The endoscope tracking system of claim 24, wherein the signal sources are spaced axially apart along a tracking zone of the endoscope.

30. The endoscope tracking system of claim 24, wherein the signal sources generate a signal in response to an input signal carried through the endoscope.

31. The endoscope tracking system of claim 24, wherein the signal sources generate a constant signal.

32. The endoscope tracking system of claim 24, wherein the signal sources generate a signal in response to receipt of an interrogator signal.

33. The endoscope tracking system of claim 32, wherein the interrogator signal source is carried by the support.

34. The endoscope tracking system of claim 32, wherein the interrogator signal source is carried remotely from the support.

35. A method of displaying the location of a medical device within a body, comprising the steps of:
introducing a medical device into the body;
propagating at least a first signal from the medical device;
receiving the signal by a receiver outside of the body; and
displaying an indicium of the signal;
wherein the displaying step comprises displaying an indicium which corresponds with the actual location of the origin of the propagated signal in approximately a 1:1 size relationship, at least in the x and y axes.

36. A method of displaying the location of a medical device as in claim 35, wherein the display does not display z axis location information.

37. A method of displaying the location of a medical device as in claim 35, wherein the displaying step comprises displaying the indicium on a flexible pad.

38. A method of displaying the location of a medical device as in claim 35, wherein the flexible pad in contact with the body.

39. A method of displaying the location of a medical device as in claim 35, wherein the displaying step comprises displaying the indicium on a display that is positioned remotely from the body.

40. A detection device, for detecting the location of a signal source within the body, comprising a support having a plurality of detectors thereon, such that a first plurality of detectors generate a first signal indicative of the proximity of the signal source, which is distinguishable from any signal generated from a second plurality of detectors which are more remote from the source.

41. A detection device, for detecting the location of a signal source within the body as in claim 40, wherein the first signal is a visible signal.

42. A detection device, for detecting the location of a signal source within the body as in claim 40, further comprising a display carried by the support for displaying the first signal.
